(19) United States
(12) Patent Application Publication
(10) Pub. No.: US 2017/0164930 A1
(43) Pub. Date: Jun. 15, 2017

(54) ULTRASOUND APPARATUS, CONTROLLING METHOD THEREOF AND TELEMEDICINE SYSTEM

(71) Applicant: SAMSUNG ELECTRONICS CO., LTD., Suwon-si (KR)

(72) Inventors: Du-seon OH, Suwon-si (KR); Hwan SHIM, Suwon-si (KR); Hyung-joon LIM, Seoul (KR)

(21) Appl. No.: 15/361,203

(22) Filed: Nov. 25, 2016

(30) Foreign Application Priority Data
Dec. 15, 2015 (KR) 10-2015-0179464
Mar. 30, 2016 (KR) 10-2016-0038212

(51) Int. Cl. A61B 8/08; A61B 8/00

(52) U.S. Cl. CPC A61B 8/5223; A61B 8/4483; A61B 8/5207; A61B 8/56; A61B 8/463; A61B 8/0883

(57) ABSTRACT

Disclosed herein is an ultrasound apparatus, a control method thereof, and a telemedicine system, the ultrasound apparatus including an ultrasound transducer configured to receive a reflected ultrasound signal and a signal processor configured to generate an ultrasound image using a first frequency band signal of the received ultrasound signal, and to generate additional information using a second frequency band signal of the received ultrasound signal.

### CROSS-REFERENCE TO RELATED APPLICATIONS

This application is based on and claims priority under 35 U.S.C. §119 to Korean Patent Applications Nos. 10-2015-0179464 and 10-2016-0038212, filed on Dec. 15, 2015 and Mar. 30, 2016, respectively, in the Korean Intellectual Property Office, the disclosures of which are incorporated by reference herein in their entireties.

### BACKGROUND

Field

Apparatuses and methods of the disclosure relate generally to an ultrasound apparatus and a controlling method thereof, and for example, to an ultrasound apparatus capable of obtaining an ultrasound image and an auscultation sound at the same time, and a controlling method thereof.

Description of Related Art

An ultrasound diagnosis system radiates ultrasound signals being generated from a transducer of a probe to an object, and obtains an image of inside the object using information received in the transducer of an ultrasound apparatus.

The ultrasound diagnosis system has non-invasive and non-destructive characteristics, and is thus capable of providing in real time a high resolution image of an object for diagnosis without having to perform an incision surgery on the object and observing the same. Due to such stability, the ultrasound diagnosis system is in wide use in the medical field.

The ultrasound diagnosis system may also be used when examining hearts. When conducting an ultrasound diagnosis on a heart, electrocardiogram (ECG) information is further required. In the case of extracting images of a cycle of the heart, it is difficult to understand the cycle of the heart with only ultrasound images, and thus the ECG information is used additionally. Accordingly, users have to go through the inconvenience of mounting equipment for ECG measurement when conducting the ultrasound diagnosis. For example, there is the inconvenience for users to attach an electrode for ECG measurement to a body part.

Further, conventional ultrasound apparatuses require a sensor configuration capable of auscultating a heart sound, and thus there occurs a problem of increased volume and weight. Such a necessity for additional equipment acts as an obstacle in realizing a portable or telemedicine diagnosis system.

### SUMMARY

An example aspect of the present disclosure is to address the aforementioned problems by providing an ultrasound apparatus capable of obtaining an ultrasound image and heart sound information at the same time using a signal received in a transducer of an ultrasound apparatus, and a controlling method thereof.

According to an example embodiment of the present disclosure, an ultrasound apparatus is provided including an ultrasound transducer configured to receive a reflected ultrasound signal; and a signal processor configured to generate an ultrasound image using a first frequency band signal of the received ultrasound signal, and to generate additional information using a second frequency band signal of the received ultrasound signal.

According to another example embodiment of the present disclosure, a method for controlling an ultrasound apparatus is provided, the method including receiving a reflected ultrasound signal; generating an ultrasound image using a first frequency band signal of the received ultrasound signal; and generating additional information using a second frequency band signal of the received ultrasound signal.

According to another example embodiment of the present disclosure, a telemedicine system is provided including an ultrasound apparatus configured to process an ultrasound signal reflected in a certain position of a first user and to transmit the processed ultrasound signal; an external apparatus configured to photograph the first user using the ultrasound apparatus, to generate an ultrasound image using the ultrasound signal transmitted from the ultrasound apparatus, and to display a photographed image of the first user and the generated ultrasound image; a telemedicine apparatus configured to receive an input of a second user; and a server configured to transceive data between the external apparatus and the telemedicine apparatus, wherein the external apparatus is configured to transmit the ultrasound image and the photographed image of the first user to the telemedicine apparatus through the server, and the telemedicine apparatus is configured to transmit input data of the second user regarding the received ultrasound image to the external apparatus through the server, and the external apparatus is configured to display the ultrasound image including the received input data of the second user.

According to the aforementioned various example embodiments of the present disclosure, it is possible to obtain information on an ultrasound image and on an auscultation sound with signals received in an ultrasound transducer itself. Further, the ultrasound apparatus according to the present disclosure and a controlling method thereof requires no further equipment, and enables easy manipulation, and may thus be useful in tele-medical treatments.

### BRIEF DESCRIPTION OF THE DRAWINGS

The above and/or other aspects of the disclosure will be more apparent from the following detailed description, taken in conjunction with the accompanying drawings, in which like reference numerals refer to like elements, and wherein:

FIG. 1A is a diagram illustrating an example ultrasound diagnosis system according to an example embodiment of the present disclosure;

FIG. 1B is a diagram illustrating an example ultrasound diagnosis system according to another example embodiment of the present disclosure;

FIGS. 2A and 2B are diagrams illustrating an example of an ultrasound apparatus according to an example embodiment of the present disclosure;

FIG. 2C is a diagram illustrating an example portable ultrasound apparatus according to an example embodiment of the present disclosure;

FIG. 3 is a block diagram illustrating an example configuration of an ultrasound apparatus according to an example embodiment of the present disclosure;

FIGS. 4A, 4B, 4C, 5A, 5B, 5C and 5D are diagrams illustrating an example ultrasound transducer according to an example embodiment of the present disclosure;

FIG. 6 is a block diagram illustrating an example configuration of an ultrasound apparatus according to an example embodiment of the present disclosure;

FIG. 7 is a diagram illustrating an example screen where an ultrasound image and ECG information are displayed at the same time;

FIG. 8 is a diagram illustrating example similarities between an ECG signal and a PCG signal;

FIG. 9 is a diagram illustrating an example screen where an ultrasound image and heart sound information are displayed at the same time;

FIG. 10 is a diagram illustrating example information for extracting a heart ultrasound image of one cycle;

FIG. 11 is a diagram illustrating an example method for displaying an ultrasound image and heart sound information at the same time;

FIG. 12 is a diagram illustrating an example of how a virtual ECG signal is generated using heart sound information;

FIGS. 13 and 14 are flowcharts illustrating an example controlling method of an ultrasound apparatus according to various example embodiments of the present disclosure;

FIG. 15 is a sequence diagram illustrating an example ultrasound diagnosis system according to an example embodiment of the present disclosure; and

FIG. 16 is a sequence diagram illustrating an example ultrasound diagnosis system according to another example embodiment of the present disclosure.

### DETAILED DESCRIPTION

Hereinafter, various example embodiments of the present disclosure will be described in greater detail with reference to the accompanying drawings. In explaining the present disclosure, when it is deemed that specific explanation on a related well known technology may unnecessarily obscure the main point of the present disclosure, detailed explanation of the related well known technology may be omitted. The terms hereinafter are terms defined in consideration of the functions in the present disclosure, and may thus vary depending on the user, operation and practice, etc. Therefore, definitions should be made based on the overall contents of the description.

Terms including numerical expressions such as a first, a second and the like may be used to explain various elements, but there is no limitation thereto. These terms are used simply for the purpose of differentiating one element from another. For example, a first element may be called a second element, and similarly, a second element may be called a first element instead. The term ‘and/or’ includes a combination of a plurality of related items or one of the plurality of related items.

The terms used in this description are intended for explaining the example embodiments of the present disclosure, and not for restricting and/or limiting the present disclosure. A singular expression includes a plural expression unless clearly mentioned otherwise. In this description, terms such as ‘include’ and ‘have/has’ should be construed as designating that there are such characteristics, numbers, operations, elements, components or a combination thereof in the description, not to exclude the existence or possibility of adding one or more of other characteristics, numbers, operations, elements, components or a combination thereof.

In the present description, an ‘ultrasound image’ may refer, for example, to an image of an object obtained using ultrasound waves. The object may, for example, be a human or an animal. Examples of the object include, but are not limited to, heart, liver, lung, brain, uterus, breast, abdominal organ and blood vessel, etc.

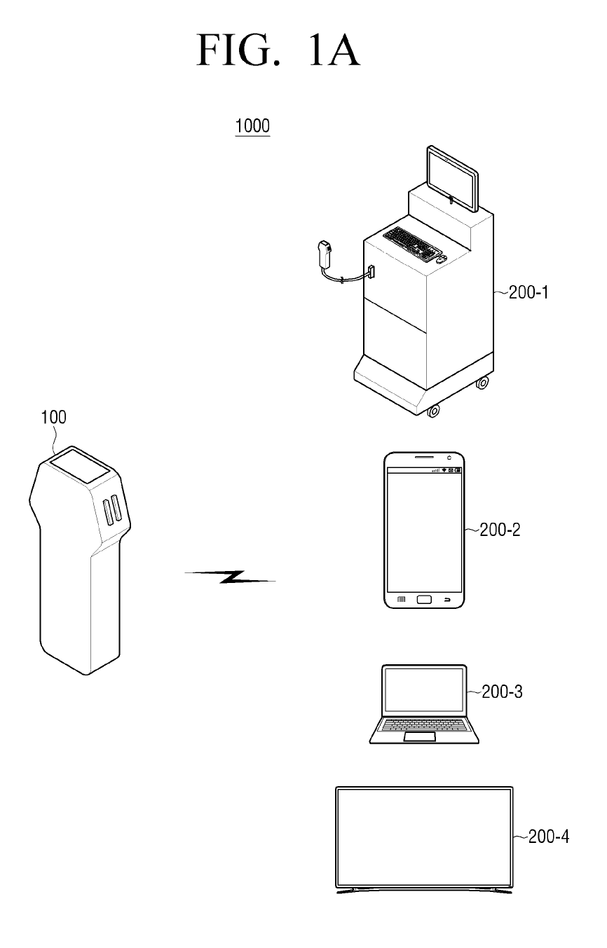

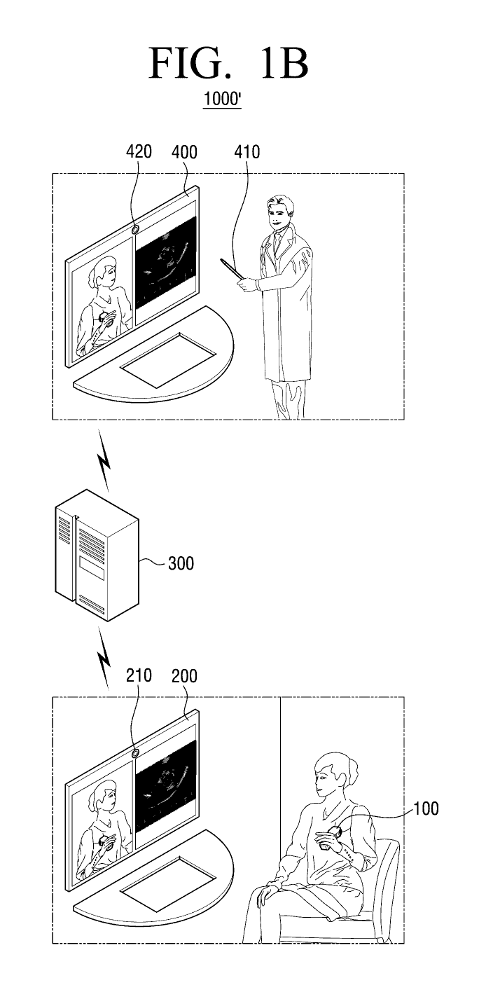

FIG. 1A is a diagram illustrating an example ultrasound diagnosis system 1000 according to an example embodiment of the present disclosure. In the embodiment of FIG. 1A, the ultrasound diagnosis system 1000 may include an ultrasound apparatus 100 and an external apparatus 200. However, the ultrasound apparatus 100 may be realized as a single apparatus that includes a display, and may perform the function of receiving ultrasound signals while the external apparatus 200 performs diagnosis functions of generating ultrasound images and the like. Since there may be such various modified example embodiments as aforementioned, the ultrasound diagnosis system 1000 is not limited to the one illustrated in FIG. 1A.

The ultrasound apparatus 100 may, for example, generate an ultrasound signal and transmit the generated ultrasound signal to the object. Further, the ultrasound apparatus 100 may receive a signal reflected by the object and generate an image of the structure or shape of the object based on the reflected signal. For example, the ultrasound apparatus 100 may transmit an ultrasound signal from a surface of the object to a certain area inside the body, and obtain an image related to a layer or blood flow of a soft tissue using information of the received ultrasound signal (also called echo signal) reflected from a body tissue.

The ultrasound apparatus 100 according to an example embodiment of the present disclosure may separate a low frequency band signal using the ultrasound signal received. The ultrasound apparatus 100 may generate an ultrasound image using a high frequency band signal or the reflected ultrasound signal itself. Further, the ultrasound apparatus 100 may separate the low frequency band signal and generate auscultation information. For example, in the case of an echocardiography examination, the ultrasound apparatus 100 may separate a low frequency band signal and generate heart sound information from the low frequency band signal. For example, because the heart sound information is included in a low frequency band of 1 kHz or below. In another example, when generating an ultrasound image of a blood vessel, the ultrasound apparatus 100 may generate information on a pulse cycle. In another example, when generating an ultrasound image of a lung, the ultrasound apparatus 100 may generate information on a respiratory cycle.

The ultrasound apparatus 100 according to another example embodiment of the present disclosure may generate auscultation information without generating an ultrasound image. For example, the ultrasound apparatus 100 realized as a portable apparatus may operate, for example, like a digital stethoscope. When an ultrasound image is unnecessary in a diagnosis, the ultrasound apparatus 100 may optionally generate the auscultation information only. In this example, the ultrasound apparatus 100 may avoid unnecessary waste of power.

Further, the ultrasound apparatus 100 may transmit at least one of the generated ultrasound image and heart sound information to the external apparatus 200. The ultrasound apparatus 100 may communicate with the external apparatus 200 wirelessly or using a wired connection.

The external apparatus 200 may display the ultrasound image and the heart sound information at the same time if necessary. The external apparatus 200 may be realized in various forms, and thus, is not limited to the ones illustrated in FIG. 1A. For example, the external apparatus 200 may be a cart type apparatus 200-1 typically used in medical institutions. In another example, the external apparatus 200 may be realized as a portable smart phone 200-2, lap-top 200-3 or the like. Further, the external apparatus 200 may be realized as a smart TV 200-4 or the like for household usage. It will be understood that the external apparatus 200 may be any suitable apparatus and is not limited to the examples listed above.

FIG. 1B is a diagram illustrating an example ultrasound diagnosis system according to another example embodiment of the present disclosure. In the embodiment of FIG. 1B, the ultrasound diagnosis system 1000′ may include, for example, an ultrasound apparatus 100, an external apparatus 200, a server 300 and a telemedicine apparatus 400.

The ultrasound apparatus 100 may transmit at least one of the ultrasound image and heart sound information to the external apparatus 200. In another example, the ultrasound apparatus 100 may transmit only ultrasound measurement data to the external apparatus 200, while the external apparatus 200 generates at least one of the ultrasound image and heart sound information. For example, the external apparatus 200 may process the ultrasound image using an installed telemedicine application.

The external apparatus 200 may include a camera 210. The external apparatus 200 may photograph a user of the ultrasound apparatus 100 with the camera 210. Further, the external apparatus 200 may display the received ultrasound image and the photographed image at the same time. Further, the external apparatus 200 may transmit at least one of the ultrasound image, the heart sound information and the photographed image of the user of the ultrasound apparatus 100 to the server 300.

The server 300 may transmit data received from the external apparatus 200 to the telemedicine apparatus 400 at the doctor's (e.g., diagnosing) side. In another example, the external apparatus 200 may transmit the data directly to the telemedicine apparatus 400 connected via network without going through the server 300.

The telemedicine apparatus 400 may display at least one of the photographed image, ultrasound image and heart sound information received. For example, the telemedicine apparatus 400 may be realized as smart TV, PC, lap-top and tablet, etc., but is not limited thereto. The telemedicine apparatus 400 may include an inputter (e.g., including input circuitry) 410 and a camera 420. Further, the telemedicine apparatus 400 may transmit the data that the doctor input through the inputter 410 and the screen of the doctor photographed using the camera 410 to the server 300. The server 300 may transmit the input data and the photographed screen of the doctor to the external apparatus 200. The external apparatus 200 may transform the displayed ultrasound image by including the data input from the telemedicine apparatus 400 in the displayed ultrasound image and display the same. Further, the external apparatus 200 may display the photographed screen of the doctor together as well.

For example, the doctor may provide an explanation using the inputter 410 realized, for example, and without limitation, as a stylus pen on top of the ultrasound image displayed on the telemedicine apparatus 400. The external apparatus 200 may include such input data in the ultrasound image and display the same together with the ultrasound image.

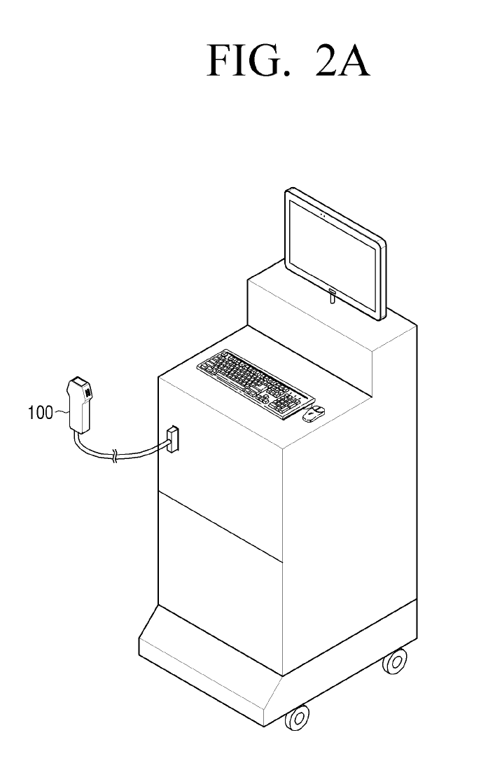

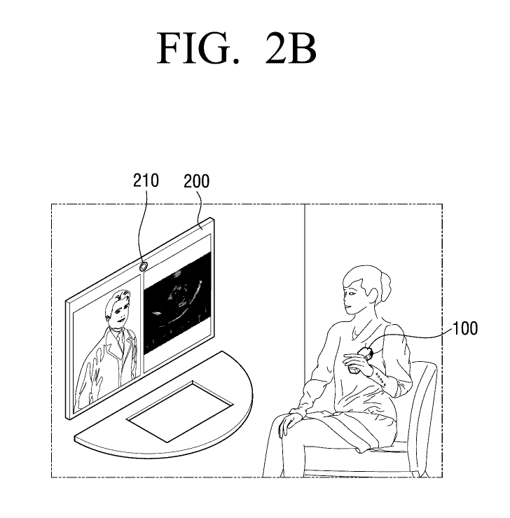

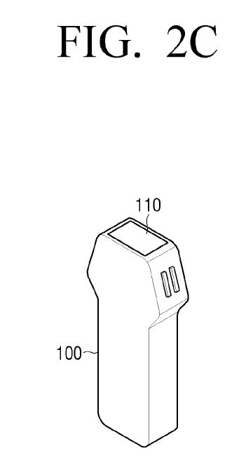

FIGS. 2A and 2B are diagrams illustrating an example concept of ultrasound apparatus 100 according to an example embodiment of the present disclosure.

FIG. 2A is a diagram illustrating the ultrasound apparatus 100 realized in a form generally used in medical institutions. In this example, the ultrasound apparatus 100 of FIG. 1 and the external apparatus 200 are connected by wire. Using the ultrasound apparatus 100 according to the embodiment of FIG. 2A, a patient who is the subject of diagnosis and a user (for example, doctor, medical image professional and clinical technologist, etc.) of the ultrasound apparatus 100 may meet and conduct an ultrasound diagnosis. In the ultrasound apparatus 100, not only a display for displaying an ultrasound image but also an inputter (e.g., including input circuitry) for manipulating operations on the ultrasound image may be realized.

In another example embodiment, it is also possible to provide perform remote treatment using the ultrasound apparatus 100 as illustrated, for example, in FIG. 2B. According to the example embodiment illustrated in FIG. 2B, the patient who is the subject of diagnosis may also be the user of the ultrasound apparatus 100. The ultrasound apparatus 100 may generate at least one of an ultrasound image and heart sound information using the ultrasound signal received. Further, the ultrasound apparatus 100 may transmit at least one of the generated ultrasound image and heart sound information generated. For example, the ultrasound apparatus 100 may transmit at least one of the ultrasound image and the heart sound information to a hospital server or another medical apparatus connected, for example, via a picture archiving and communication system (PACS). The patient who is the user of the ultrasound apparatus 100 may check the ultrasound image being displayed on the external apparatus 200, and receive explanation on the diagnosis results via communication with the doctor.

According to an example embodiment of the present disclosure, the external apparatus 200 may photograph the user of the ultrasound apparatus 100 using the camera 210. Further, the external apparatus 200 may analyze the photographed image and determine the measured body area of the ultrasound image. When the measured body area is determined, the external apparatus 200 may incorporate the location information on the determined measured area in the ultrasound image. For example, the external apparatus 200 may be configured such that the location information is automatically tagged to the ultrasound image. In the example embodiment of FIG. 2B, the external apparatus 200 may analyze the image of the user photographed by the camera 210 and determine that the measured body area is the left breast. By including such information in the ultrasound image, it is possible to identify that the ultrasound image is, for example, that of the heart.

FIG. 2B illustrates an example where the external apparatus 200 is realized as a smart TV, but there is no limitation thereto. For example, it is possible to transmit an ultrasound signal of a high frequency band and an ultrasound signal of a low frequency band received and separated in the ultrasound apparatus 100 to a smart phone, and the smart phone may generate an ultrasound image and heart sound information with the ultrasound signal received through an application for ultrasound diagnosis. Further, the smart phone may display the generated ultrasound image and the heart sound information and transmit the same to a medical institution at the same time. A result of diagnosis conducted by the medical institution may be transmitted back to the smart phone and provided to the user together with the ultrasound image.

The ultrasound apparatus 100 according to an example embodiment of the present disclosure may be realized as a portable apparatus as illustrated, for example, in FIG. 2C. The ultrasound apparatus 100 realized as the portable apparatus including a transducer 110 and may be useful in remote treatments and emergency medical situations.

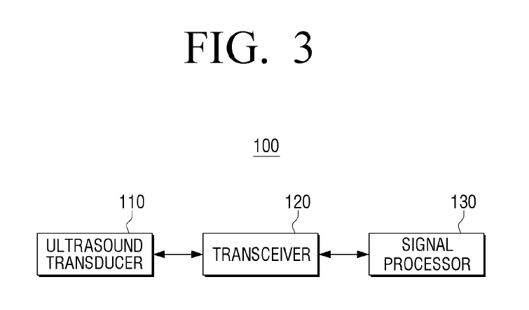

FIG. 3 is a block diagram illustrating an example ultrasound apparatus 100 according to an example embodiment of the present disclosure. Referring to FIG. 3, the ultrasound apparatus 100 may include an ultrasound transducer 110, a transceiver 120 and a signal processor 130.

The ultrasound transducer 110 may emit ultrasound waves to the object in order to obtain an image of inside the object, and receive an ultrasound signal reflected from the object. The ultrasound transducer 110 may include, for example, a plurality of piezoelectric vibrators. Through the plurality of piezoelectric vibrators, the ultrasound transducer 110 may convert an electrical signal being supplied from the transceiver 120 into dynamic vibrational energy to generate ultrasound waves, and convert vibrations received from outside back into electrical signals.

The transceiver 120 may transmit the electrical signal to the ultrasound transducer 110, and receive an electrical signal of the ultrasound signal received from the ultrasound transducer 110. For example, the transceiver 120 may be realized as a multi-channel transceiver that enables signal transmission through a plurality of channels (e.g., transmission channels).

The signal processor 130 may use the ultrasound signal received from the ultrasound transducer 110 to generate at least one of an ultrasound image and additional information. For example, the signal processor 130 may generate the ultrasound image using a signal of a first frequency band of the ultrasound signal received. Further, the signal processor 130 may generate additional information using a second frequency band signal of the ultrasound signal received. The additional information may, for example, include heart sound information, respiratory sound information and pulse sound information and the like depending on the measurement object.

The signal processor 130 according to an example embodiment of the present disclosure may separate the received ultrasound signal into a first frequency band signal and a second frequency band signal. For example, the signal processor 130 may separate the received ultrasound signal into a high frequency band signal of 3.5 MHz and a low frequency band signal of 20˜1000 Hz.

Further, the signal processor 130 may convert the separated first frequency band signal (high frequency band signal) into a digital signal. The signal processor 130 may generate an ultrasound image using the converted digital signal. For example, the signal processor 130 may generate a B mode (bright mode), an M mode (motion), or a D mode (doppler mode) ultrasound image.

In another example embodiment, when generating the ultrasound image, the signal processor 130 may omit the operation of separating the ultrasound signal into a high frequency band signal, and generate the ultrasound image using the received ultrasound signal itself. That is because if the low frequency band signal does not have a great effect, it is efficient not to perform the frequency band separating operation in the signal processor 130.

The signal processor 130 may, for example, perform a beam-forming on the separated second frequency band signal (low frequency band signal). For example, the signal processor 130 may perform analog beam-forming and amplification on the second frequency band signal (low frequency band signal). The low frequency band signal is typically relatively weaker than the high frequency band signal, and thus it may be desirable to perform the analog beam-forming before converting the low frequency band signal into a digital signal.

Further, the signal processor 130 may convert the beam-formed second frequency band signal (low frequency band signal) into a digital signal. The signal processor 130 may generate additional information using the converted digital signal. For example, the additional information may include graph information representing information on heart sound (for example, the size of the heart sound and spectrum, etc.) by time.

According to the aforementioned various example embodiments of the present disclosure, it is possible to obtain an auscultation sound using only the ultrasound transducer 110 without having a separate sensor or microphone configured to obtain auscultation sounds. Hereinafter, heart ultrasound waves and heart sound will be explained as representative examples, but the present disclosure may of course be applied to any kind of ultrasound diagnosis method that uses ultrasound waves and auscultation sound signals at the same time.

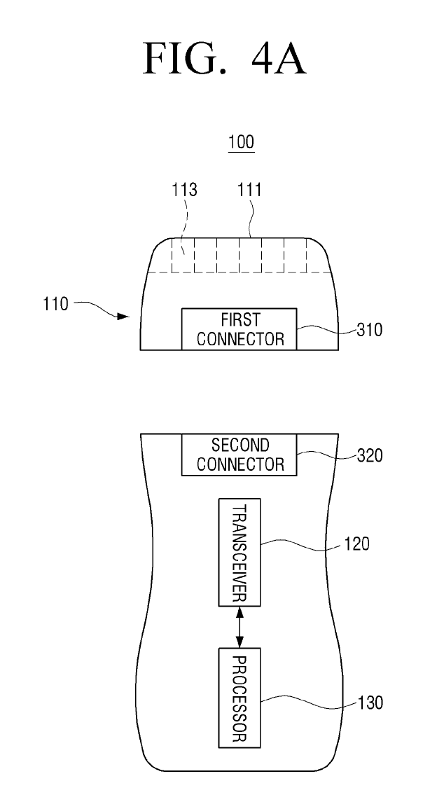

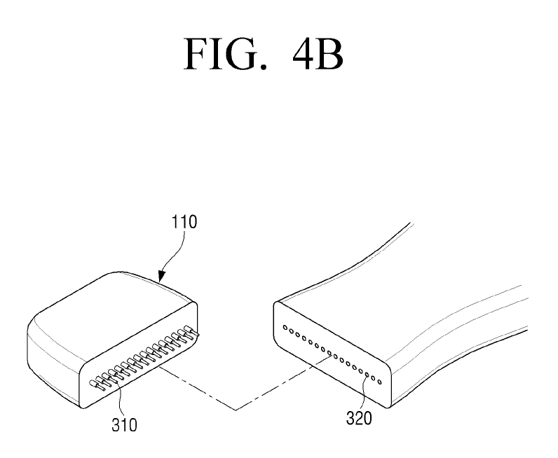

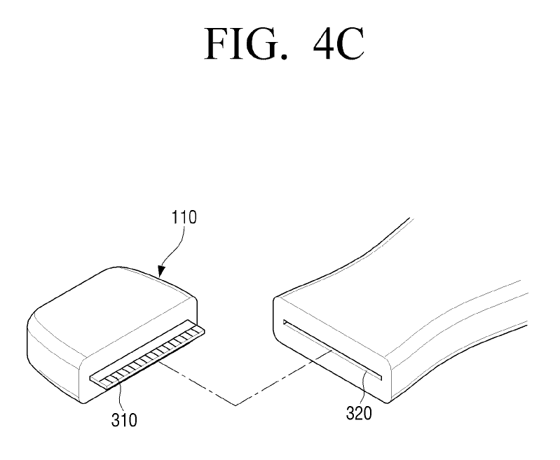

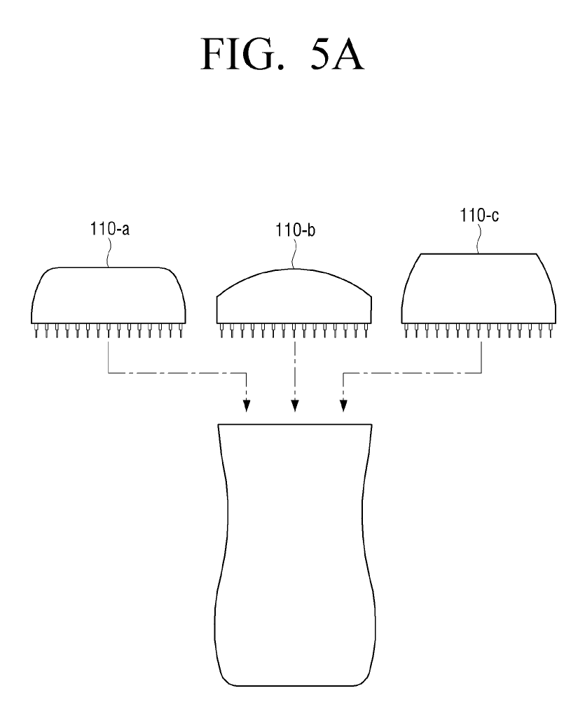

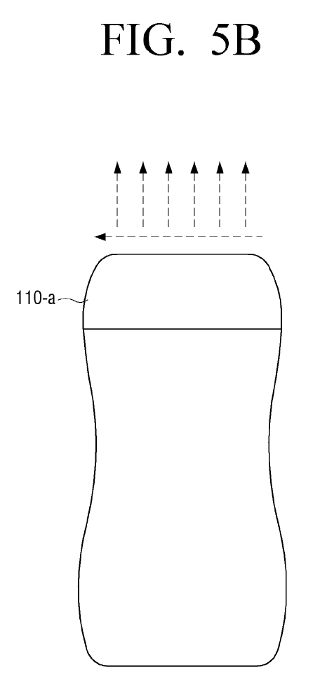

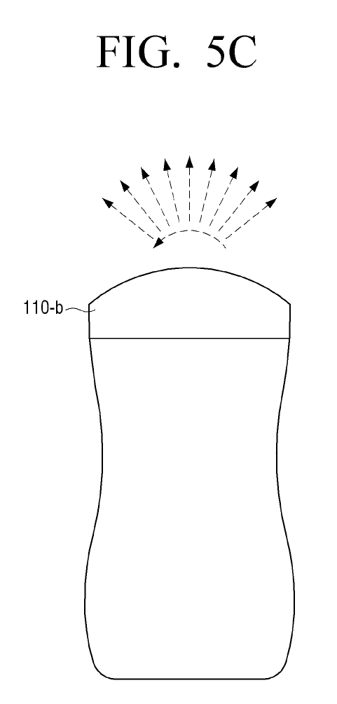

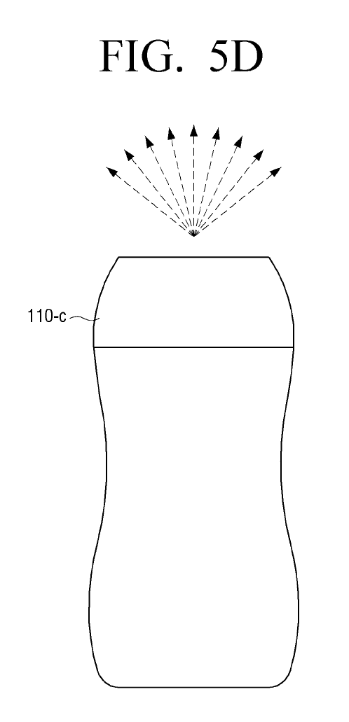

FIGS. 4A, 4B, 4C, 5A, 5B, 5C and 5D are diagrams illustrating examples of the ultrasound transducer 110 according to an example embodiment of the present disclosure.

Referring to FIG. 4A, the ultrasound transducer 110 may include a transducer array 111 including a plurality of conversion elements 113 for mutually converting electrical signals and sound signals arranged in certain forms. For example, the transducer array 111 may be realized in various forms such as a piezoelectric transducer that uses the piezoelectric effect of piezoelectric material, a capacitive micromachined transducer that mutually converts ultrasound waves and electrical signals using changes in capacitance, a magnetic micromachined transducer that mutually converts ultrasound waves and electrical signals using changes in a magnetic field, and an optical transducer that mutually converts ultrasound waves and electrical signals using changes in optical characteristics, or the like, but is not limited thereto.

For example, the conversion elements 113 may be formed by dividing a piezoelectric material into a plurality of pieces. The piezoelectric material may, for example, include a piezoelectric ceramic, a single crystalline, and polymer compound or the like that cause the piezoelectric phenomenon.

The plurality of conversion elements 113 may be arranged in various forms to form the transducer array 111. For example, the plurality of conversion elements 113 may be arranged in a linear array, or in a convex array, or the like. Additionally or alternatively, the plurality of conversion arrays 113 may be arranged in a plurality of layers (phased array). The transducer array 111 may include a cover (not shown) that covers the upper portion of the plurality of arranged conversion elements 113.

The transducer array 111 may be realized, for example, as a one dimensional array or two dimensional array. The case where the plurality of conversion elements 113 are arranged in one dimension on a plane vertical to the proceeding direction of the ultrasound waves is called the one dimensional transducer array. Further, the case where the plurality of conversion elements 113 are arranged in two dimension on the plane vertical to the proceeding direction of the ultrasound waves is called the two dimensional transducer array.

The two dimensional transducer array may transmit the ultrasound waves to the object along an external scan line by appropriately delaying the input time of the signals being input into each conversion element 113. Further, since it is possible to obtain a three-dimensional image using a plurality of echo signals, configuring a two dimensional transducer array is well-suited to realizing a three dimensional image.

Referring to FIG. 4A, the ultrasound apparatus 100 according to an example embodiment of the present disclosure may be configured such that a portion that includes the ultrasound transducer 110 and a portion that includes the transceiver 120 and the signal processor 130 are detachable. An electrical connecting or releasing of the ultrasound transducer 110 and the signal processor 130 may be performed, for example, by combining or separating a first connector 310 and a second connector 320.

The first connector 310 may enable the electrical signal of the ultrasound transducer 110 to be transmitted to the transceiver 120. The first connector 310 may be disposed in an area opposite to the area where the transducer array 111 is disposed, and a portion of the first connector 310 may be exposed outside the transducer 110. By the exposed portion, the first connector 310 and the second connector 320 may be connected. The first connector 310 and the second connector 320 may include a conductive material capable of transmitting signals.

FIGS. 4B and 4C illustrate examples in which the ultrasound transducer 110 portion and the rest of the portion including the transceiver 120 are connected in the ultrasound apparatus 100 according to an example embodiment of the present disclosure.

Referring to FIG. 4B, the first connector 310 may include a plurality of protrusions (e.g., pins). Further, the second connector 320 may include a plurality of grooves (e.g., holes) corresponding to the plurality of protrusions of the first connector 310. The plurality of protrusions and grooves may each be made of a conductive material. As the plurality of protrusions are inserted into the plurality of grooves, the first connector 310 and the second connector 320 may be connected. In this manner, the ultrasound transducer 110 may be electrically connected to the transceiver 120 and the signal processor 130.

In another example, as illustrated in FIG. 4C, the first connector 310 may be realized in a plug form, and the second connector 320 may be realized in a jack form configured to receive and connect to the first connector 310.

According to an example embodiment of the present disclosure, the ultrasound transducer 110 may be realized in various forms depending on the purpose of usage. The ultrasound transducer 110 may be realized in various forms depending on the arrangement form of the conversion elements 113 that form the transducer array 111. Referring to FIG. 5A, the ultrasound transducer 110 may be realized in a linear array 110-a, convex array 110-b, or phased array 110-c. In the case of the phased array ultrasound transducer 110-c, the plurality of conversion elements 113 may be arranged in dual layers or multiple layers.

The user may selectively use various forms of ultrasound transducers 110-a, 110-b, 110-c depending on the purpose of usage of the ultrasound apparatus 100 (for example, according to the body part that is the subject of diagnosis).

FIG. 5B is a diagram illustrating an example in which the ultrasound transducer 110 is realized as a linear array ultrasound transducer 110-a according to an example embodiment of the present disclosure. The linear ultrasound transducer 110-a may provide a result of diagnosis on a body part having a shallow depth in high resolution using frequency of, for example, 3˜8 MHz. For example, the linear array transducer 110-a may be used in diagnosing on breasts, thyroid and musculoskeleton system etc. However, the depth and frequency of the diagnosis may vary depending on the standard of the transducer.

FIG. 5C illustrates an example in which the ultrasound transducer 110 according to an example embodiment of the present disclosure is realized as a convex array ultrasound transducer 110-b. The convex array ultrasound transducer 110-b may observe a deep body part using the frequency of, for example, 2˜5 MHZ. For example, the convex array ultrasound transducer 110-b may be utilized in diagnosing abdominal parts in ob-gyn evaluations, and the like.

FIG. 5D illustrates an example in which the ultrasound transducer 110 is realized as a phased array ultrasound transducer 110-c according to an example embodiment of the present disclosure. The phased array ultrasound transducer 110-c is capable of observing body parts such as those having narrow gaps therebetween, for example, between ribs. Further, the phased array ultrasound transducer 110-c may be used to diagnose hearts.

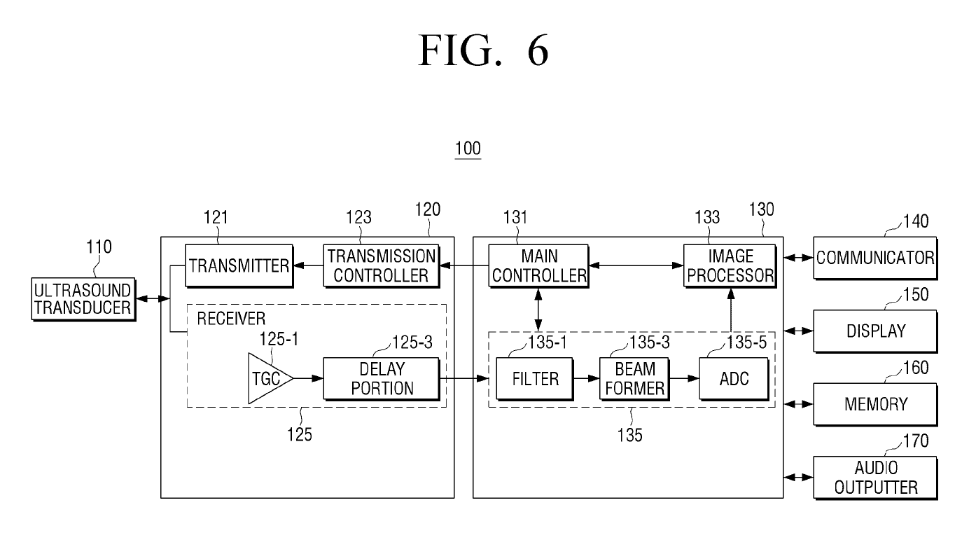

FIG. 6 is a block diagram illustrating an example ultrasound apparatus 100 according to an example embodiment of the present disclosure. Referring to FIG. 6, the ultrasound apparatus 100 may include an ultrasound transducer 110, a transceiver 120, a signal processor 130, a communicator (e.g., including communication circuitry) 140, a display 150, a memory 160 and an audio outputter (e.g., including audio output circuitry) 170. However, the ultrasound apparatus 100 according to an example embodiment of the present disclosure may not necessarily include the display 150 or the audio outputter 170. Further, the ultrasound apparatus 100 may of course further include a configuration not illustrated in the example embodiment of FIG. 6.

The ultrasound transducer 110 may contact the surface of the object and emit ultrasound waves to the object. Further, the ultrasound transducer 110 may receive an ultrasound signal reflected by the object.

For example, the ultrasound transducer 110 may be realized in a transducer array form including of a plurality of transducers. For example, the ultrasound transducer 110 may be a multichannel transducer. Each of the transducers may be equipped, for example, with a piezoelectric vibrator to generate the ultrasound waves from electrical signals, and convert the ultrasound signals back into electrical signals.

The ultrasound transducer 110 according to an example embodiment of the present disclosure may, for example, be a piezo-electric transducer (PZT), but is not limited thereto. For example, the ultrasound transducer 110 may alternatively be realized as a capacitive Micromachined Ultrasonic Transducer (cMUT), or the like, but is not limited thereto.

In the example embodiment of FIG. 6, the transceiver 120 may include, for example, a transmitter 121, a transmission controller 123 and a receiver 125. Further, the receiver 125 may include a TGC (Time Gain Compensating circuit) 125-1 and a delay portion 125-3.

The transmitter 121 may supply a driving signal to the ultrasound transducer 110. The ultrasound transducer 110 may emit ultrasound waves to the object by generating vibrations in response to the driving signal.

The transmission controller 123 may generate a driving signal based on the electrical signal received from the main controller 131 of the signal processor 130. The transmission controller 123 may convert the digital signal received from the main controller 131 into an analog signal and transmit the analog signal to the transmitter 121.

The transmission controller 123 may generate a pulse for forming a transmission ultrasound signal according to pulse repetition frequency PRF. Further, the transmitter 121 may apply a delay time for determining transmission directionality to the pulse. Each pulse to which the delay time is applied corresponds to each piezoelectric vibrator forming the ultrasound transducer 110.

Based on a control of the transmission controller 123, the transmitter 121 may supply a driving signal to the ultrasound transducer 110 at a timing corresponding to each pulse to which the delay time is applied.

The receiver 125 may include a TGC 125-1 and a delay portion 125-3. The time gain compensation unit (TGC) (e.g., including time gain compensation circuitry) 125-1 may amplify the ultrasound signals received from the ultrasound transducer 110 and compensate time gain. The TGC is a parameter for compensating the received ultrasound signals (echo signals) by attenuating according to the depth of diagnosis. When using the ultrasound signals that passed the TGC 125-1, it is possible to generate ultrasound images with improved screen quality even when diagnosing deep areas.

The delay portion 125-3 may apply a delay time for determining the reception directionality to the received ultrasound signals (echo signals). The delay portion 125-3 may allow ultrasound data to be generated by summing the signals to which the delay time is applied. For example, the delay portion 125-3 may process the signals such that an ultrasound reflectance component of a certain direction is emphasized.

The signal processor 130 may include a main controller 131, an image processor 133, a filter 135-1, a beam former (e.g., including beam forming circuitry) 135-3, and an analog to digital converter (ADC) 135-5, etc.

The main controller 131 may control the overall operations of the ultrasound apparatus 100. The main controller 131 may load a program used as a volatile memory (for example, RAM) from a nonvolatile memory (for example, ROM) where the programs is stored, and execute the program. For example, the main controller 131 may boot the system using an operating system O/S stored in the ROM or the like inside the signal processor 130 or the memory 160. Further, the main controller 131 may perform various operations using various programs, applications, contents, data and the like stored in the memory 160.

The filter 135-1 may use various filter circuitry configured to separate the received ultrasound signal into a high frequency band signal and a low frequency band signal. The received ultrasound signal may include a plurality of electrical signals converted by the plurality of piezoelectric vibrators forming the ultrasound transducer 110. For example, the filter 135-1 may separate the received ultrasound signal into a high frequency band signal and a low frequency band signal using filter circuitry, such as, for example, and without limitation, a high pass filter (HPF) and a low pass filter (LPF), respectively.

The beam former 135-3 may include beam forming circuitry configured to focus a predetermined low frequency band signal of the received ultrasound signal. For example, the beam former 135-3 may adjust the phase of each of the plurality of electrical signals in an analog beam forming method and focus the same into one signal. This is because the time it takes for a reflected ultrasound signal to approach the plurality of piezoelectric vibrators forming the ultrasound transducer 110 may all be different. For example, the beam former 135-3 may apply the delay time for determining the reception directionality to the reflected ultrasound signal. Further, the beam former 135-3 may sum each of the signals to which the delay time is applied.

Meanwhile, the beam former 135-3 may focus a predetermined high frequency band signal of the received ultrasound signal. The operations of the beam former 135-3 may be the same as in focusing the low frequency band signal. However, the beam former 135-3 may not necessarily perform a focusing in the high frequency band.

The ADC 135-5 may convert ultrasound data of an analog form into digital data. For example, the ADC 135-5 may perform conversion at a high speed on the ultrasound signals of a high frequency band, but perform conversion at a low speed on the ultrasound signals of a low frequency band.

The image processor 133 may generate an ultrasound image using the ultrasound signal. The ultrasound signal used for generating the ultrasound image may, for example, be the received ultrasound signal itself or the ultrasound signal of the first frequency band (for example, the predetermined high frequency band). For example, the image processor 133 may generate the ultrasound image through a scan conversion process on the ultrasound signal of the high frequency band processed into a digital signal. The image processor 125 may generate at least one of an image of A mode (amplitude mode), B mode (brightness mode), C mode (color mode), D mode (doppler mode) and M mode (motion mode).

Further, the image generator 133 may generate a three dimensional ultrasound image, and may add additional information to the ultrasound image in the form of a text, a graphic, etc.

According to an example embodiment of the present disclosure, the signal processor 130 may generate the additional information using a signal of the second frequency band (for example, low frequency band signal). The additional information may be graph information representing changes in the heart sound over time. The additional information may, for example, include information representing changes in respiratory sound, pulse sound, etc.

For example, in the case where the additional information is the heart sound information, the signal processor 130 may detect a first heart sound corresponding to a starting time of a contraction period of the heart and a second heart sound corresponding to a starting time of a relaxation period of the heart and generate the information. Using the locations of the first heart sound and the second heart sound on a time axis, the signal processor 130 may extract a heart ultrasound image of, for example, one cycle.

The signal processor 130 may generate virtual ECG information using a corresponding relationship between the heart sound information and ECG. Explanation of the corresponding relationship between the heart sound information and the ECG will be explained in greater detail below.

The communicator 140 may include various communication circuitry configured to transmit at least one of the ultrasound image and the additional information to the external apparatus 200. The communicator 140 may transmit the ultrasound image or the additional information using a wired or wireless method.

For example, the communicator 140 may use various wireless communication circuitry implementing various wireless communication methods such as the near field communication (NFC) method, the wireless LAN method, the infrared (IR) communication method, the Zigbee communication method, the WiFi method and the Bluetooth method, etc. Further, the communicator 140 may use various wired communication circuitry implementing various wired communication methods such as the high definition multimedia interface (HDMI) method, the low voltage differential signaling (LVDS) method, the local area network (LAN) method and the universal serial bus (USB) method, etc.

The communicator 140 may transceive data to and from a hospital server or another medical apparatus in the hospital through a medical image information system (PACS). Further, the communicator 140 may, for example, perform data communication according to a digital imaging and communications in medicine (DICOM) standard.

The display 150 may display the ultrasound image generated. Further, the display 150 may display various additional information such as the heart sound information and the ECG information that may be processed in the ultrasound apparatus 100, together with the ultrasound image.

The memory 160 may store various information to be processed by the ultrasound apparatus 100. For example, the memory 160 may store the received ultrasound signal, the ultrasound image, the heart sound information, and the ECG information, etc. The memory 160 refers, for example, to a storage medium for storing various programs and the like necessary for operating the ultrasound apparatus 100. The memory 160 may be realized as a flash memory, a hard disk, etc., but is not limited thereto. For example, the memory 160 may be equipped with a ROM for storing programs for performing operations of the ultrasound apparatus 100, and a RAM for temporarily storing the data according to operations of the ultrasound apparatus 100. Further, the memory 160 may be further equipped with an electrically erasable and programmable ROM (EEPROM) and the like for storing various reference data.

The audio outputter 170 may include various audio output circuitry configured to output an auscultation sound such as the heart sound generated in the ultrasound apparatus 100. Further, the audio outputter 170 may output guideline messages related to manipulating the ultrasound apparatus 100. For example, in the case of a telemedicine treatment, the user may manipulate the ultrasound apparatus 100 according to received voice guidelines explaining the method of using the ultrasound apparatus 100 being output from the audio output circuitry of the audio outputter 170.

The aforementioned, the ultrasound apparatus 100 may further include a user inputter (e.g., including input circuitry, not illustrated) and a wireless discharger (not illustrated), etc.

The user inputter may include various input circuitry configured to receive an input for controlling the ultrasound apparatus 100 from the user. For example, the user inputter may include various input circuitry, such as, for example, and without limitation, a key pad, a mouse, a touch panel, a touch screen, a track ball, and a jog switch, etc. Further, the user inputter may be realized as a voice recognition sensor, a fingerprint recognition sensor and a motion recognition sensor, etc., but is not limited thereto

A wireless charger (not shown) may charge the power of the ultrasound apparatus 100 using, for example, a magnetic induction method or a self-resonant method. The magnetic induction method refers, for example, to a technology of loading a resonant coil of a same frequency on a charging pad or a charging object, and using resonance to load power in a frequency. Especially, in the case where the ultrasound apparatus 100 is realized as a portable device, the ultrasound apparatus 100 may include a wireless charger. However, even in the case where the ultrasound apparatus 100 is realized as a portable device, the ultrasound apparatus 100 may also be realized in other various methods such as wired charging method, method of supplying power using battery and the like, and thus the power charging method of the ultrasound apparatus 100 is not limited to wireless charging methods.

The ultrasound apparatus 100 according to an example embodiment of the present disclosure may be used in echocardiography examinations related to heart diseases. In an echocardiography examination, the size and function of the heart, thickness of the heart wall, valves of the heart, ischemic heart disease and the like may be diagnosed. The ultrasound apparatus 100 may, for example, use at least one mode of B mode, M mode and D mode in order to make such diagnoses.

B mode refers, for example, to a method for indicating a reflected ultrasound signal using a brightness of a dot. The brightness of each dot may be proportional to an amplitude of the reflected ultrasound signal. For example, the ultrasound apparatus 100 may generate a B mode image using 256 or more brightness levels.

M mode is a method for indicating a distance of a moving reflector using temporal variations.

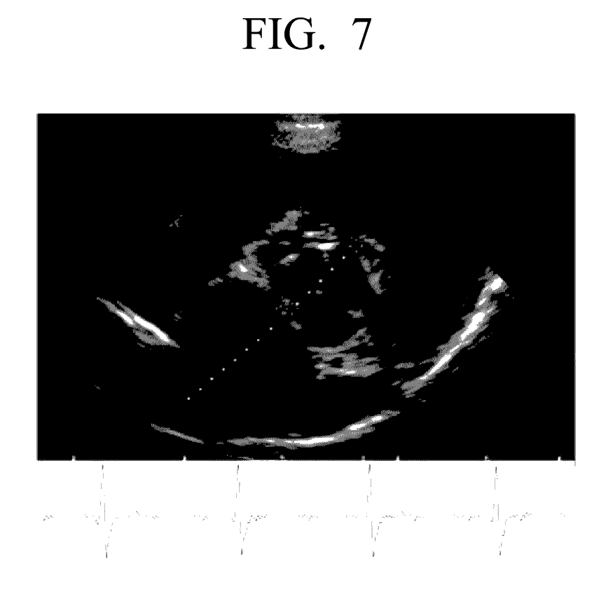

Further, in an echocardiography examination, the ECG may be additionally measured in all cases. FIG. 7 is a diagram illustrating an example screen of the ultrasound apparatus where the ultrasound image and the ECG information of M mode are displayed together.

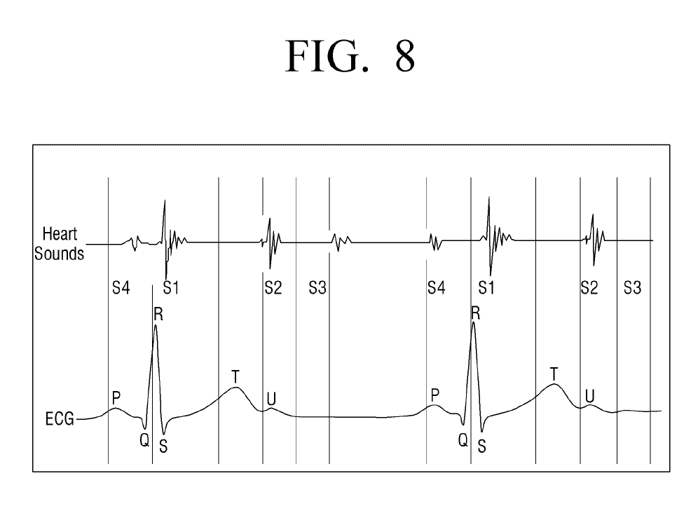

The ultrasound apparatus 100 according to an example embodiment of the present disclosure may obtain a criteria of time axis regarding an ultrasound image using a phonocardiogram (PCG) signal instead of the ECG signal. As illustrated in FIG. 8, the ECG signal and the PCG signal have a corresponding relationship. The graph at the upper end of FIG. 8 is a graph illustrating heart sounds by time axis. Further, the graph at the lower end of FIG. 8 is a graph illustrating the electrocardiogram (ECG) by time axis.

There were attempts in the past as well to obtain heart sounds taking notice of the similarities between heart sounds and ECG. However, according to such conventional methods, additional configurations were needed to measure heart sounds, and there are no known attempts made to extract heart auscultation sounds from a ultrasound signal such as in the ultrasound apparatus 100 according to the example embodiments of the present disclosure. For example, in the past, attempts were made to measure heart sounds by measuring PCG using microphones or by measuring vibrations using acceleration sensors. Therefore, since additional equipment was necessary, the conventional methods cannot be regarded to have advantages compared to the method of using ECG according to the present disclosure.

The ultrasound apparatus 100 according to the example embodiments of the present disclosure are different from such conventional ultrasound apparatuses in that both an ultrasound image and additional information (for example, heart sound information) may be obtained using the ultrasound signal received from the ultrasound transducer 110.

The signal processor 130 may generate additional information (for example, heart sound information) using the second frequency band signal of the received ultrasound signals. For example, the second frequency band signal may be a signal of the 20˜1000 Hz frequency band, but is not limited thereto.

In an example where the additional information is heart sound information, a signal of a low frequency band from which the heart sound information may be extracted may generally have a weaker signal intensity compared to a high frequency band signal, and thus the signal processor 130 may intensify a low frequency band signal by performing analog beam forming. Further, the signal processor 130 may convert the low frequency band signal into a digital signal to generate heart sound information.

The ultrasound apparatus 100 according to an example embodiment of the present disclosure may be realized as one that includes or does not include a display 150. For example, in the case where the ultrasound apparatus 100 does not include a display 150, the ultrasound apparatus 100 may transmit the generated ultrasound image and additional information to the external apparatus 200. In another example, in the case where the ultrasound apparatus 100 includes a display 150, the ultrasound apparatus 100 itself may display the ultrasound image and additional information at the same time.

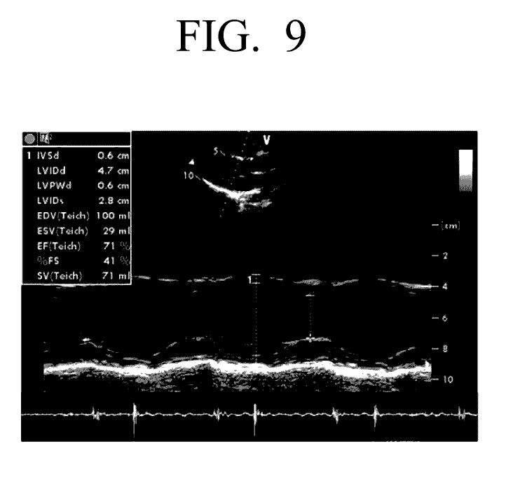

FIG. 9 is a diagram illustrating an example screen in which an ultrasound M mode image and heart sound information are displayed at the same time according to an example embodiment of the present disclosure. In FIG. 9, the image in the center is the ultrasound M mode image, and the graph by time axis at the lower end is the heart sound information. Further, not only the ultrasound M mode image and heart sound information, but also additional information may be displayed together in a text form (left upper corner of FIG. 9).

The ultrasound apparatus 100 according to an example embodiment of the present disclosure may generate an ultrasound image and heart sound information using only the received ultrasound signal as illustrated in FIG. 9. A cycle of the heart may be identified based on the heart sound information, and thus, the ultrasound apparatus 100 may automatically extract an image corresponding to one cycle of the heart.

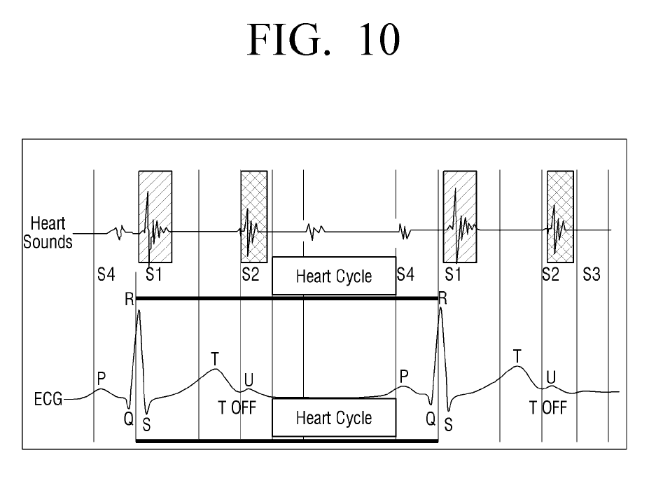

FIG. 10 is a diagram illustrating an example method for extracting a heart ultrasound image of one cycle in the ultrasound apparatus according to an example embodiment of the present disclosure. Referring to FIG. 8, the first heart sound S1 represents a starting time point of a contraction period of the heart generated by a QRS signal of ECG. The second heart sound S2 represents a starting time point of a relaxation period of the heart generated by a T signal of ECG.

For example, the first heart sound S1 section may, for example, be an audio signal generated by a flow of blood generated as a contraction period starts. The time point of the first heart sound may correspond to the R peak of ECG. Further, the second heart sound S2 section may, for example, be an audio signal generated by a flow of blood and opening/closing of the heart valve generated as a relaxation period starts. The time point of the second heart sound may correspond to the T OFF point of ECG.

Since the signal cycle of ECG and the signal cycle of PCG are the same as the heart cycle as mentioned above, the ultrasound apparatus 100 according to an example embodiment of the present disclosure may extract PCG from the ultrasound signal to substitute for ECG.

Further, the first heart sound and the second heart sound may be used as reference points when observing heart valves during contraction and relaxation of the heart necessary in echocardiography. The first heart sound section of the heart sound information extracted from the ultrasound apparatus 100 may, for example, be the section where the heart contracts, and thus from the ultrasound image corresponding to the first heart sound section, a mitral valve and a tricuspid valve may be observed. Further, the second heart sound section of the heart sound information extracted from the ultrasound apparatus 100 may, for example, correspond to the section where the heart relaxes, and thus from the ultrasound image corresponding to the second heart sound section, an aortic valve and a pulmonary valve may be observed.

The signal processor 130 according to an example embodiment of the present disclosure may control the display 150 to detect the first heart sound and the second heart sound from the heart sound information and to display information of a time point when the first heart sound and the second heart sound were generated.

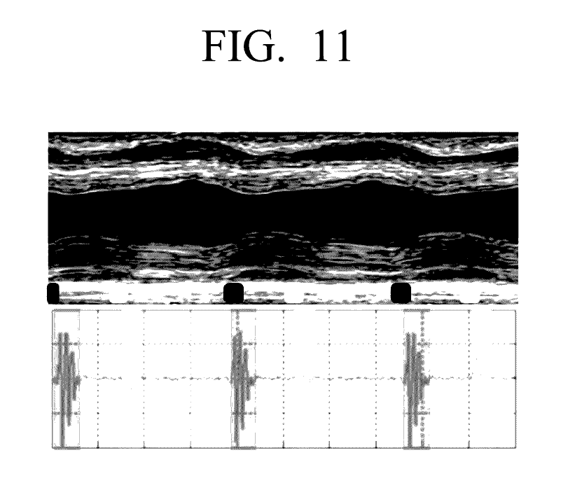

FIG. 11 is a diagram illustrating an example method for displaying, by the ultrasound apparatus 100, an ultrasound image and heart sound information at the same time, according to an example embodiment of the present disclosure. As in the upper end screen of FIG. 11, the ultrasound apparatus 100 may display an ultrasound image separated by the first heart sound section and the second heart sound section together with the ultrasound image. In another example, the ultrasound apparatus 100 may display all the graphs corresponding to the heart sound information as in the lower end screen of FIG. 11, and may display the first heart sound section and the second heart sound section such that they are highlighted.

Main users of the ultrasound apparatus 100 may be used to the ECG information and the ultrasound image being displayed together. The ultrasound apparatus 100 according to an example embodiment of the present disclosure may convert the heart sound information into ECG information using the information on the time point where the first heart sound and the second heart sound are generated.

For example, the ultrasound apparatus 100 may store a reference ECG wave form in the memory 160. Further, the ultrasound apparatus 100 may generate a virtual ECG wave form such that it corresponds to the heart sound information by setting a parameter of the reference ECG wave form. The ultrasound apparatus 100 may display the ultrasound image and the virtual ECG wave form together. The ultrasound apparatus 100 according to an example embodiment of the present disclosure may provide an ultrasound image screen that is used to the user using PCG corresponding to the ECG without actually measuring the ECG.

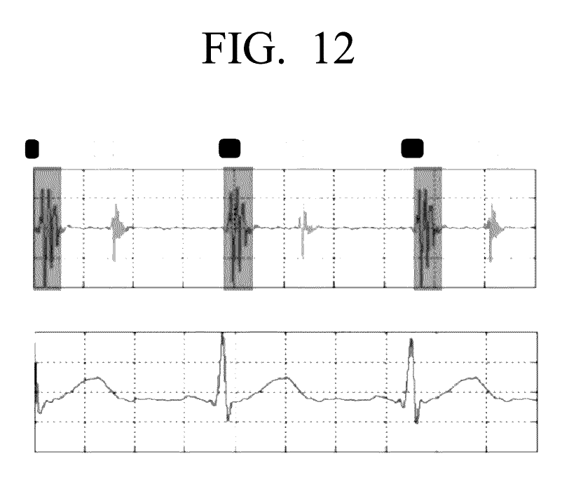

FIG. 12 is a diagram illustrating an example of how a virtual ECG information is generated using the heart sound information in the ultrasound apparatus 100 according to an example embodiment of the present disclosure. The graph at the upper end of FIG. 12 represents the heart sound information, and the graph located at the lower end represents the virtual ECG information. The ultrasound apparatus 100 may, for example, have the first heart sound correspond to R peak and have the second heart sound correspond to the T OFF point, thereby generating the virtual ECG information.

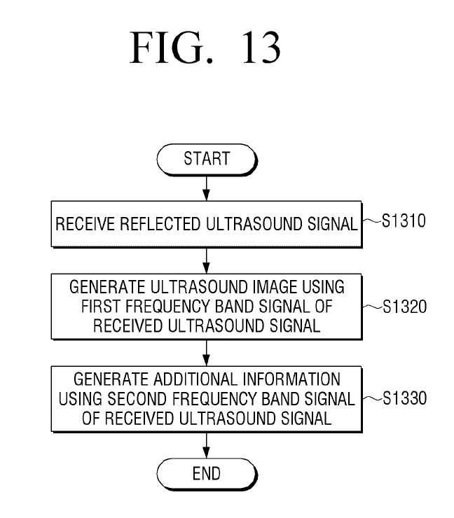

FIG. 13 is a flowchart illustrating an example method of controlling the ultrasound apparatus 100 according to an example embodiment of the present disclosure. Referring to FIG. 13, the ultrasound apparatus 100 may transmit an ultrasound signal to an object. Further, the ultrasound apparatus 100 may receive an ultrasound signal reflected by the object (S1310).

For example, the ultrasound apparatus 100 may generate an ultrasound image of the object using a signal of the first frequency band of the ultrasound signal received (S1320). For example, the ultrasound apparatus 100 may separate the high frequency band signal from the received ultrasound signal and use the separated high frequency band signal in generating the ultrasound image. In another example, the ultrasound apparatus 100 may use the received ultrasound signal itself to generate the ultrasound image. That is because, in the case where the signal of the second frequency band is small or weak enough to disregard, compared to the signal of the first frequency band, a process of separating the signal of the first frequency band may be unnecessary.

Further, the ultrasound apparatus 100 may generate additional information using the second frequency band signal of the received ultrasound signal (S1330). For example, the additional information may be graph information representing information of heart sound by time axis. The heart sound information may be realized in various forms such as the size of the heart sound and spectrum information, etc., but is not limited thereto.

The aforementioned steps S1320 and S1330 may, for example, be performed at the same time in parallel, or step S1330 may be performed prior to step S1320.

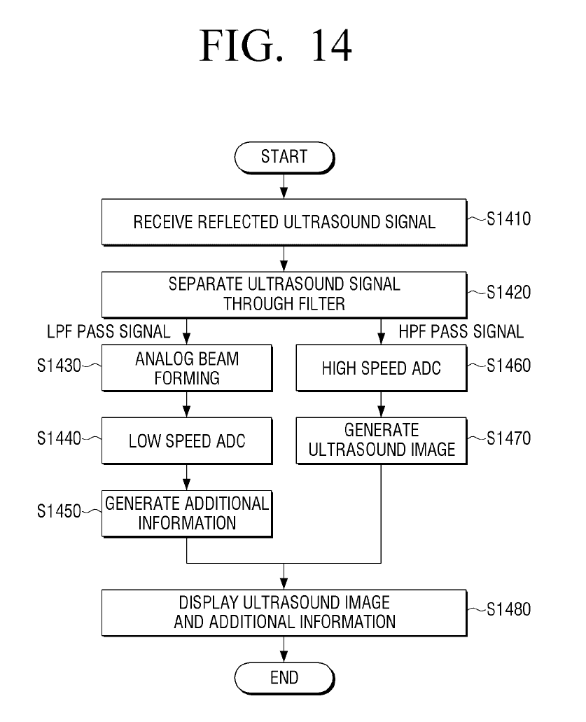

FIG. 14 is a flowchart illustrating an example method of controlling the ultrasound apparatus 100 according to another example embodiment of the present disclosure. In the example embodiment of FIG. 14, it will be explained that the received ultrasound signal is separated into a low frequency band and a high frequency band and then each signal is processed in parallel, but there is no limitation thereto.

The ultrasound apparatus 100 may transmit an ultrasound signal to an object. Further, the ultrasound apparatus 100 may receive the ultrasound signal reflected by the object (S1410). Further, the ultrasound apparatus 100 may separate the ultrasound signal by frequency band through a filter (S1420). For example, the ultrasound apparatus 100 may separate the predetermined low frequency band signal of the received ultrasound signal. The predetermined low frequency band may be, for example, 20˜1000 Hz band. The low frequency band signal is used to generate additional information such as the heart sound information. For example, the additional information may be the auscultation information on the object.

Further, the ultrasound apparatus 100 may separate the predetermined high frequency band signal of the received ultrasound signal through the high pass filter (HPF). The predetermined high frequency band may be 3.5 MHz band. The high frequency band signal may be used, for example, to generate the ultrasound image.

For example, the ultrasound apparatus 100 may perform an analog beam forming on the ultrasound signal (low frequency band signal) that passed the LPF (S1430). The low frequency band signal may be focused by the analog beam forming and intensified. Further, the ultrasound apparatus 100 may convert the focused low frequency band signal into a digital signal through, for example, low speed ADC (S1440). The ultrasound apparatus 100 may generate additional information using the low frequency band signal converted into the digital signal (S1450). For example, the additional information may be auscultation information on the object.

Further, the ultrasound apparatus 100 may convert the ultrasound signal (high frequency band signal) that passed the HPF into a digital signal through a high speed ADC (S1460). In another example, the ultrasound apparatus 100 may perform an analog beam forming on a high frequency band signal. However, the analog beam forming on the high frequency band signal does not necessarily have to be performed. The ultrasound apparatus 100 may generate an ultrasound image using the high frequency band signal converted into a digital signal (S1470).

Such an ultrasound apparatus 100 may generate an ultrasound image and additional information using only the received ultrasound signal. For example, in the case where the additional information is the heart sound information, the ultrasound apparatus 100 may generate virtual ECG information using the heart sound information.

The ultrasound apparatus 100 may display the generated ultrasound image and the additional information (S1480). When necessary, the ultrasound apparatus 100 may change the information included in the display screen and display the same to the user. For example, the ultrasound apparatus 100 may display only the ultrasound image, or display the ultrasound image, the heart sound information and the virtual ECG information at the same time.

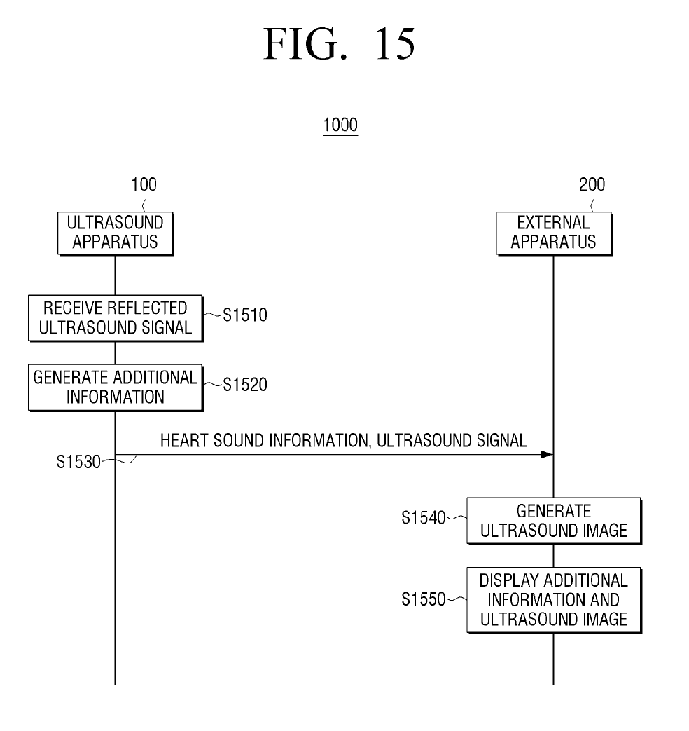

FIG. 15 is a sequence diagram illustrating example operations of an ultrasound diagnosis system according to an example embodiment of the present disclosure. The ultrasound apparatus 100 according to the example embodiment of FIG. 15 may generate only the heart sound information by itself, and transmit the ultrasound signal processed into a digital signal to the external apparatus 200. Further, in the external apparatus 200, an ultrasound image may be generated using the ultrasound signal.

For example, the ultrasound apparatus 100 may receive the ultrasound signal reflected by the object (S1510). Further, the ultrasound apparatus 100 may generate additional information using the received ultrasound signal (S1320). For example, the ultrasound apparatus 100 may separate the low frequency band signal from the ultrasound signal, and process the separated low frequency band signal to generate heart sound information. The heart sound information is an example of the additional information that may be generated by the ultrasound apparatus 100 when the object is a heart.

The ultrasound apparatus 100 may transmit the generated additional information and the received ultrasound signal to the external apparatus 200 (S1530). The external apparatus 200 may generate an ultrasound image using the ultrasound signal received from the ultrasound apparatus 100 (S1540). Further, the external apparatus 200 may display the generated ultrasound image together with the received heart sound information (S1550).

The ultrasound apparatus 100 according to another example embodiment may generate by itself both the heart sound information and the ultrasound image. The ultrasound apparatus 100 according to another example embodiment may only serve the function of separating the received ultrasound signal into a high frequency band signal and a low frequency band signal.

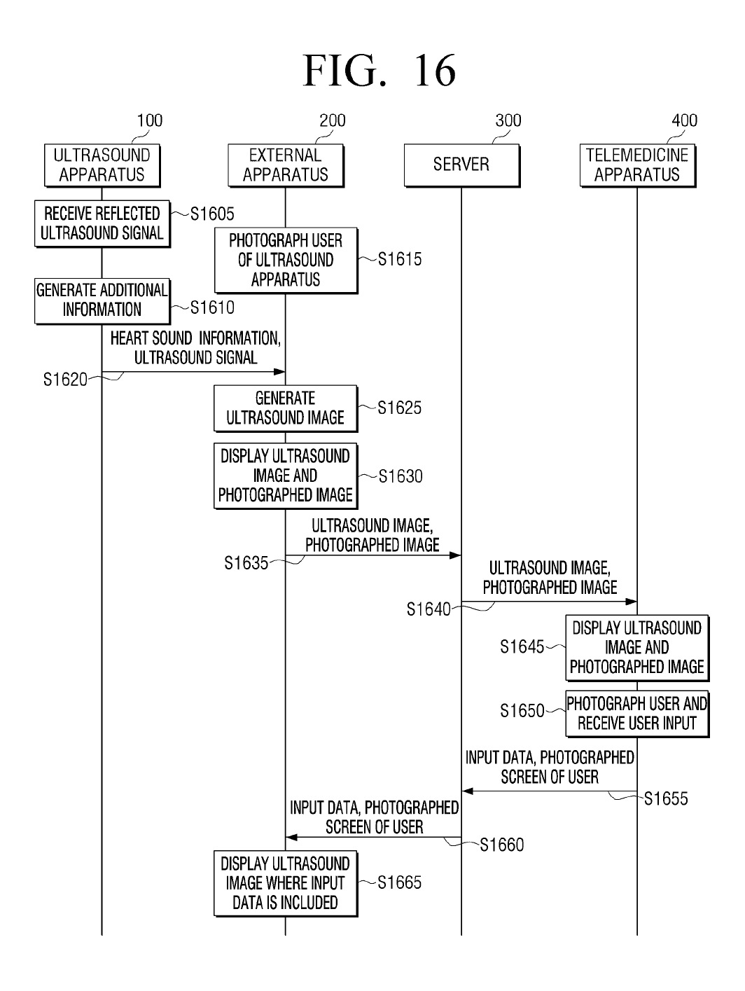

FIG. 16 is a sequence diagram illustrating example operations of the ultrasound diagnosis system according to another example embodiment. Steps S1605, S1610 and S1620 are the same or similar to steps S1510, S1520 and S1530, respectively, and thus repeated explanation will be omitted.

The external apparatus 200 may photograph the user of the ultrasound apparatus 100 using a camera equipped therein or connected thereto (S1615). Further, the external apparatus 200 may generate an ultrasound image using the received ultrasound signal (S1625). Further, the external apparatus 200 may display the received heart sound information together with the ultrasound image. Further, the external apparatus 200 may display the received ultrasound image and the photographed image at the same time (S1630). For example, the external apparatus 200 may analyze the photographed image and determine of which part the ultrasound measurement is being performed. Further, the external apparatus 200 may incorporate the location information on the determined body part measured in the ultrasound image.

The external apparatus 200 may transmit at least one of the ultrasound image and the photographed image of the user of the ultrasound apparatus 100 to the server 300 (S1635). The server 300 may transmit the data received from the external apparatus 200 to the telemedicine apparatus at the telemedicine apparatus located at the doctor's side 400 (S1640). Of course, however, the external apparatus 200 may transmit the data to the telemedicine apparatus 400 connected via network without going through the server 300.

The telemedicine apparatus 400 may display at least one of the received photographed image, the ultrasound image and the heart sound information (S1645). Further, the telemedicine apparatus 400 may photograph the doctor who is the user of the telemedicine apparatus 400, and receive data input by the doctor (S1650). The telemedicine apparatus 400 may transmit a screen photographed by the doctor to the server 300 using the data that the doctor input through the inputter and the camera (S1655). The server 300 may transmit the input data and the photographed screen of the doctor to the external apparatus 200 (S1660).

The external apparatus 200 may display an ultrasound image in which the received input data is included (S1665). The external apparatus 200 may display the photographed screen of the doctor together with the ultrasound image.

The methods explained above may be realized in forms of program commands that may be performed through various computer means, and may then be recorded in computer readable media. The computer readable media may include a program command, data file, or data structure, or a combination thereof. The program commands that may be recorded in the computer readable media may be those specially designed and configured for the present disclosure or those well known to one skilled in the art and thus made available. Examples of the computer readable record media include magnetic media such as hard disks, floppy disks and magnetic tape, optical media such as CD-ROMs and DVDs, magneto-optical media such as floptical disks and hardware devices such as ROMs, RAMs and floppy memory specially configured to store and perform program commands. Examples of the program commands include not only machine codes that are made by compilers but also high-level codes that may be executed by computers using interpreters and the like. The hardware devices mentioned above may be configured to operate as one or more software in order to perform the operations of the present disclosure, and vice versa.

The foregoing example embodiments and features are merely examples and are not to be construed as limiting the disclosure. The present teaching can be readily applied to other types of apparatuses. Also, the description of the example embodiments is intended to be illustrative, and not to limit the scope of the claims, and many alternatives, modifications, and variations will be apparent to those skilled in the art.

What is claimed is:

1. An ultrasound apparatus comprising:
an ultrasound transducer configured to receive a reflected ultrasound signal; and
a signal processor configured to generate an ultrasound image using a first frequency band signal of the received ultrasound signal, and to generate additional information using a second frequency band signal of the received ultrasound signal.

2. The ultrasound apparatus according to claim 1,
wherein the signal processor includes beam former circuitry configured to focus the second frequency band signal of the received ultrasound signal, and
the signal processor is further configured to generate the additional information using the second frequency band signal focused by the beam former circuitry.

3. The ultrasound apparatus according to claim 1,
wherein the signal processor comprises:
a filter configured to separate the received ultrasound signal into a first frequency band signal and a second frequency band signal; and
an analog-digital converter (ADC) configured to convert the received ultrasound signal into a digital signal.

4. The ultrasound apparatus according to claim 1,
wherein the additional information comprises graph information representing information on heart sound by time.

5. The ultrasound apparatus according to claim 1,
further comprising communication circuitry configured to transmit the ultrasound image and the additional information to an external apparatus.

6. The ultrasound apparatus according to claim 1,
further comprising a display configured to display the ultrasound image and the additional information.

7. The ultrasound apparatus according to claim 6,
wherein, when the additional information comprises heart sound information, the signal processor is configured to detect a first heart sound representing a starting time point of a contraction period of a heart and to detect a second heart sound representing a starting time point of a relaxation period of the heart from the heart sound information, and to control a display to display information at a time point where the first heart sound and the second heart sound are generated.

8. The ultrasound apparatus according to claim 7,
wherein the signal processor is configured to convert the heart sound information into electrocardiogram (ECG) information using the information at a time point where the first heart sound and the second heart sound are generated.

9. The ultrasound apparatus according to claim 8,
wherein the signal processor is configured to control the display to display the ultrasound image and the electrocardiogram (ECG) information together.

10. The ultrasound apparatus according to claim 1,
wherein the second frequency band signal is a signal in a range of a 20˜1000 Hz frequency band.

11. A method for controlling an ultrasound apparatus, the method comprising:
receiving a reflected ultrasound signal;
generating an ultrasound image using a first frequency band signal of the received ultrasound signal; and
generating additional information using a second frequency band signal of the received ultrasound signal.

12. The method according to claim 11,
further comprising:
separating the received ultrasound signal into a first frequency band signal and a second frequency band signal;
focusing the separated second frequency band signal; and
converting the focused second frequency band signal and the separated first frequency band signal into a digital signal.

13. The method according to claim 11,
wherein the additional information comprises graph information representing information on a heart sound by time.

14. The method according to claim 11,
further comprising transmitting the ultrasound image and the additional information to an external apparatus.

15. The method according to claim 11,
further comprising displaying the ultrasound image and the additional information.

16. The method according to claim 15,
further comprising detecting a first heart sound representing a starting time point of a contraction period of a heart and a second heart sound representing a starting time point of a relaxation period of the heart, when the additional information is heart sound information; and
displaying information on a time point where the first heart sound and the second heart sound are generated.

17. The method according to claim 16,
further comprising converting the heart sound information into electrocardiogram (ECG) information using the information at a time point where the first heart sound and the second heart sound are generated.

18. The method according to claim 17,
wherein the displaying the ultrasound image and the heart sound information displays the ultrasound image and the electrocardiogram (ECG) information together.

19. A telemedicine system comprising:
an ultrasound apparatus configured to process an ultrasound signal reflected in a certain position of a first user and to transmit the processed ultrasound signal;
an external apparatus configured to photograph the first user using the ultrasound apparatus, to generate an ultrasound image using the ultrasound signal transmitted from the ultrasound apparatus, and to display a photographed image of the first user and the generated ultrasound image;
a telemedicine apparatus configured to receive an input of a second user; and
a server configured to transceive data between the external apparatus and the telemedicine apparatus,
wherein the external apparatus is configured to transmit the ultrasound image and the photographed image of the first user to the telemedicine apparatus through the server, and
the telemedicine apparatus is configured to transmit input data of the second user to the external apparatus through the server, and
the external apparatus is configured to display the ultrasound image including the received input data of the second user.

20. The telemedicine system according to claim 19,
wherein the external apparatus is configured to analyze the photographed image of the first user and to determine a measurement position, and to incorporate information of the determined measurement position in the ultrasound image.
